(19) United States
(12) Patent Application Publication
(10) Pub. No.: US 2012/0259143 A1
(43) Pub. Date: Oct. 11, 2012

(54) FLUOROALKYLALKOXYLATES

(75) Inventors: Kenneth Gene Moloy, Hockessin, DE (US); Sheng Peng, Hockessin, DE (US); Sourav Kumar Sengupta, Wilmington, DE (US)

(73) Assignee: E.I. DU PONT DE NEMOURS AND COMPANY, Wilmington (DE)

(21) Appl. No.: 13/081,556

(22) Filed: Apr. 7, 2011

(51) Int. Cl. C07C 43/13

(52) U.S. Cl. 568/615

(57) ABSTRACT

Disclosed is a fluorinated alkylalkoxylate compound of Formula 1,

R_{f}—O—(CF₂)ₓ(CH₂)_{y}—O—(QO)_{z}—H (1)

wherein
R_{f} is a linear or branched perfluoroalkyl having 1 to 6 carbon atoms optionally interrupted by one to three ether oxygen atoms;
x is an integer of 1 to 6;
y is an integer of 1 to 6;
Q is a linear 1,2-alkylene group of the formula CₘH₂ₘ where m is an integer of 2 to 10; and
z is an integer of 1 to 30.

### FIELD OF THE INVENTION

This invention relates to a fluorinated alkylalkoxylate, and a method for its preparation in which a fluorinated alcohol is contacted with an alkylene epoxide in the presence of boron-based catalysts.

### BACKGROUND

Materials containing alcohol alkoxylate have been used in a wide variety of industrial applications, for example as nonionic surfactants. They are typically prepared by the reaction of an alcohol with an alkylene epoxide such as ethylene oxide (i.e., oxirane) or propylene oxide (i. e., 2-methyloxirane) in the presence of one or more catalysts. Fluorinated alkylalkoxylates which are prepared by the reaction of an alcohol incorporating a fluorinated alkyl group with an alkylene epoxide are an important class of materials. Fluorinated alkylalkoxylates are especially useful in several industrial applications, including use as nonionic surfactants in various areas including the manufacture of polyvinylchloride (PVC) films, electrochemical cells, and various photographic and other coatings.

Known catalyst systems and methods for the alkoxylation of fluorinated alcohols include Lewis acids such as boron trifluoride or silicon tetrafluoride, and basic catalysts derived from metal hydrides, alkyls or alkoxides. Unfortunately, the Lewis acidic materials also catalyze side reactions such as dimerization of alkylene epoxides to form dioxanes during the alkylalkoxylation. The use of strong bases as catalysts alone, such as those used commercially for the manufacture of nonfluorinated alkoxylated alcohols, is not satisfactory for alkoxylation of fluorinated alcohols and results in undesirable elimination of fluoride and formation of olefin impurities:

R_{f}CF₂CH₂CH₂OH+base R_{f}CF═CHCHOH+F⁻

U.S. Patent Publication 2010/0280280 describes a process for preparation of a fluorinated alkyloxylate in which at least one fluorinated alcohol is contacted with at least one alkylene epoxide in the presence of a catalyst system comprising an alkali metal borohydride, and an organic quaternary salt.

U.S. Pat. No. 5,608,116 discloses preparation of fluoralkylalkoxylates using a commercial mixture of perfluoroalkylethanols having the general structure R_{f}CH₂CH₂OH wherein R_{f} is a linear or branched perfluoroalkyl group of up to 30 carbon atoms is alkoxylated in the presence of a catalyst system comprising an iodine source and alkali metal borohydride.

It is also taught that fluorinated materials require 8 or more fluorinated carbons in the perfluoroalkyl chain to provide desirable properties. Honda et al., in Macromolecules, 2005, 38, 5699-5705 teach that for perfluoroalkyl chains of greater than or equal to 8 carbons, orientation of the perfluoroalkyl groups, designated Rf groups, is maintained in a parallel configuration while for such chains having less than 6 carbons, reorientation occurs. This reorientation decreases surface properties such as contact angle, surface tension, etc. Thus, fluorinated materials containing shorter chain perfluoroalkyls (≦6 carbons) have traditionally and in general practice not been successful commercially for providing desirable properties.

The fluorinated materials derived from long chain perfluoroalkyl groups having 8 or more carbons are expensive. It is desirable to maintain or improve surface effects (surface tension, leveling, blocking, etc.) and to increase the fluorine efficiency; i.e., boost the efficiency or performance of treating agents so that lesser amounts of the expensive fluorinated composition are required to maintain or even improve performance. It is desirable to reduce the chain length of the perfluoroalkyl groups thereby reducing the amount of fluorine present, while still achieving the same or superior surface effects. Thus there is a need for a method using a catalyst system which provides desirable reactivity in the alkoxylation of alcohols having short chain or partially fluorinated groups. The present invention solves the needs described above.

### BRIEF SUMMARY OF THE INVENTION

The present invention related to a compound of Formula 1:

R_{f}—O—(CF₂)ₓ(CH₂)_{y}—O—(QO)_{z}—H (1)

wherein R_{f} is a linear or branched perfluoroalkyl having 1 to 6 carbon atoms, optionally interrupted by one to three ether oxygen atoms,
x is an integer of 1 to 6;
y is an integer of 1 to 6;
Q is a linear 1,2-alkylene group of the formula CₘH₂ₘ where m is an integer of 2 to 10; and
z is an integer of 1 to 30.

The present invention further comprises compounds where R_{f} is C₃F₇ or CF₃CF₂CF₂.

The present invention further comprises compounds where x is 2.

The present invention further comprises compounds where y is 2.

The present invention further comprises compounds where Q is CH₂CH₂ or CH₂CH(CH₃) or a mixture thereof.

### DETAILED DESCRIPTION

All trademarks are denoted herein by capitalization.

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), or both A and B are true (or present).

As used herein, the phrase “one or more” is intended to cover a non-exclusive inclusion. For example, one or more of A, B, and C implies any one of the following: A alone, B alone, C alone, a combination of A and B, a combination of B and C, a combination of A and C, or a combination of A, B, and C.

Also, use of “a” or “an” are employed to describe elements and described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the disclosed compositions, suitable methods and materials are described below.

In case of conflict, the present specification, including definitions, will control. In addition, the materials, methods, and examples are illustrative only and not intended to be limiting.

In the foregoing specification, the concepts have been disclosed with reference to specific embodiments. However, one of ordinary skill in the art appreciates that various modifications and changes can be made without departing from the scope of the invention as set forth in the claims below.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all embodiments.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

The concepts disclosed herein will be further described in the following examples, which do not limit the scope of the invention described in the claims.

The present invention discloses a fluorinated alkylalkoxylate of Formula (1)

R_{f}—O—(CF₂)ₓ(CH₂)_{y}—O—(QO)_{z}—H (1)

wherein

R_{f} is a linear or branched perfluoroalkyl having 1 to 6 carbon atoms, optionally interrupted by one to three oxygen atoms;

x is an integer of 1 to 6;

y is an integer of 1 to 6;

Q is a linear 1,2-alkylene group of the formula CₘH₂ₘ where m is an integer of 2 to 10.

The formulation (QO)_{z} can be a mixture of oligomers where the value of z is in the range of 1 to 30 and wherein the value of z is in the range of 4 to 15 which is preferred.

The Formula (1) of the present invention can be prepared by a method in which at least one partially fluorinated alcohol containing a R_{f}—O—(CF₂)x(CH₂)y-moiety wherein R_{f}, x, and y are defined as in Formula 1 is contacted with an alkylene epoxide in the presence of a catalyst system comprising a boron compound. Details of the method of preparation are described below.

The fluorinated alkylalkoxylates of Formula (1) are especially useful in several industrial applications, including use as nonionic surfactants in the manufacture of polyvinylchloride (PVC) films, electrochemical cells, and various photographic coatings. One of the desired properties of the fluorinated alkylalkoxylates of the present invention is their ability to lower surface tension at very low concentration in aqueous media. Typically use of the compounds of Formula (1) results in surface tensions of less than 25 milli-Newtons per meter (mN/m) at 0.1% in water. This surfactant property results in uses in many aqueous media including various coatings, such as paints, stains, polishes, and other coating compositions, especially as leveling and anti-blocking agents. The compounds of the present invention are also useful in various oil field operations.

R_{f} is a short perfluoroalkyl group with no more than 6 carbon atoms. One of the advantages of the fluorinated alkylalkoxylates of the present invention is that they provide desired surface properties while increasing fluorine efficiency. By the term “fluorine efficiency” as used herein is meant the ability to use a minimum amount of fluorinated compound and lower level of fluorine to obtain the same or enhanced surface properties. The compounds of the present invention are also useful as surfactants to reduce surface tension, and have low critical micelle concentration, while having reduced fluorine content due to the partial fluorination and/or short perfluoroalkyl chain length of 6 carbons or less.

The present invention further comprises a method of altering the surface behavior of a liquid comprising adding to the liquid a compound of Formula (1) as defined above. Normal surface tension of deionized water is 72 dynes per centimeter (dynes/cm, 72 mN/m). The above compound of Formula (1) is a surfactant which lowers surface tension at a specified rate. Generally better performance is obtained at higher concentrations of the surfactant in water. The surface tension values of the surfactant in a medium, typically a liquid, are less than about 25 (mN/m), preferably less than about 21 (mN/m), at a concentration of the surfactant in the medium of less than about 0.5% by weight.

The method of the present invention includes altering surface behavior, typically for lowering surface tension and critical micelle concentration (CMC) values, in a variety of applications, such as in coatings, cleaners, oil field agents, and many other applications. Types of surface behavior which can be altered using the method of the present invention include wetting, penetration, spreading, leveling, flowing, emulsifying, dispersing, repelling, releasing, lubricating, etching, bonding, antiblocking, foaming, and stabilizing. Types of liquids which can be used in the method of the present invention include a coating composition, latex, paint, stain, polymer, floor finish, ink, emulsifying agent, foaming agent, release agent, repellency agent, flow modifier, film evaporation inhibitor, wetting agent, penetrating agent, cleaner, grinding agent, electroplating agent, corrosion inhibitor, etchant solution, soldering agent, dispersion aid, microbial agent, pulping aid, rinsing aid, polishing agent, personal care composition, drying agent, antistatic agent, floor finish, or bonding agent.

The compounds and method of the present invention are useful in a variety of applications where a low surface tension is desired, such as coating formulations for glass, wood, metal, brick, concrete, cement, natural and synthetic stone, tile, synthetic flooring, paper, textile materials, plastics, and paints. The compounds and method of the present invention are useful in waxes, finishes, and polishes to improve wetting, leveling, and gloss for floors, furniture, shoe, and automotive care. The present invention is also useful in a variety of aqueous and non-aqueous cleaning products for glass, tile, marble, ceramic, linoleum and other plastics, metal, stone, laminates, natural and synthetic rubbers, resins, plastics, fibers, and fabrics. The present invention is also useful in oil field agents used for drilling and stimulation applications.

The compounds of Formula (1):

R_{f}—O—(CF₂)ₓ(CH₂)_{y}—O—(QO)_{z}—H (1)

can be prepared by reacting a fluorinated alcohol and an alkoxylating agent. In one embodiment, a fluorinated alcohol of Formula (2), or a mixture of such fluorinated alcohols,

R_{f}—O—(CF₂)ₓ(CH₂)_{y}—OH (2)

wherein

R_{f} is a linear or branched perfluoroalkyl having 1 to 6 carbon atoms, optionally interrupted by one to three ether oxygen atoms,

x is an integer of 1 to 6;

and y is an integer of 1 to 6;

is contacted with one or more alkoxylating agents, such as an alkylene epoxide, in the presence of a catalyst system comprising (1) at least one boron-containing compound and (2) a source of iodine or bromine. Optionally, an iodine or bromine source selected from the group consisting of an alkali metal halide, an alkaline earth metal halide, an organic quaternary ammonium halide, or an elemental halide, and mixtures thereof is also present as part of the catalyst system. The catalyst system is effective in the absence of promoters or other catalysts such as strong bases, although such materials can be present if desired.

Suitable fluorinated alcohols for use as a reactant in the disclosed method are those of Formula (2) as defined above. The R_{f} in the fluorinated alcohol can be those wherein R_{f} is a perfluoroalkyl group of 1 to 6 carbons. Also alcohols wherein x is 1 to 6 and y is 1 to 6 can be used.

Any number of alkoxylating agents such as an alkylene epoxide can be used as a reactant in the disclosed method. Of these ethylene oxide(oxirane), propylene oxide(2-methyloxirane), and mixtures of these can be used. The two or more alkylene epoxides can be added as a mixture, or added sequentially. Faster reactivity can be obtained if ethylene oxide alone is used.

The contacting is performed in the presence of the catalytic system at a temperature in the range between about 90° C. and 200° C. For commercial operations a temperature of about 100° C. to about 170° C. can be used. Temperature is maintained within a suitable range by appropriate means known in the art. The method is performed at pressures of from atmospheric pressure to about 200 psig (1580×10³ Pa). Alternatively, a pressure of about atmospheric to 50 psig (446×10³ Pa), or of about 20 psig (239×10³ Pa) to about 50 psig (446×10³ Pa) can be used.

The disclosed method of preparation permits flexibility in its operation. The catalyst can be added to the fluorinated alcohol prior to or during the addition of the alkoxylating agent. Alternatively, the fluorinated alcohol is mixed with the catalyst prior to addition of the alkoxylating agent and heating.

The catalytic system used in the disclosed method is comprised of two elements as follows: (1) boron compound and (2) an iodine or bromine compound.

The boron compounds suitable for use in the catalyst system used in the disclosed method to prepare compounds of Formula (1) include sodium borohydride, sodium triethyl borohydride, potassium borohydride, lithium borohydride, boric acid, boric oxide, and boron esters such as trimethylborate and triethylborate. The mole ratio of boron compound in the catalyst to fluorinated alcohol can vary widely and is at least of about 0.005 to 1.0 or higher. The upper limit is imposed only by practical considerations such as the cost of excessive borohydride use, contamination of product and waste streams with excess boron, and potential difficulty in controlling the rate of the exothermic alkoxylation reaction. The mole ratio can be of about 0.005:1.0 to about 0.25:1.0. The optimum mole ratio of boron to fluorinated alcohol will be affected by such factors as the structures of the fluorinated alcohol and alkoxylating agent, and the temperature, pressure and cooling efficiency of the reaction vessel. For the reaction of an alkylene oxide, such as ethylene oxide, propylene oxide, butylene oxide, styrene oxide and the like, with fluorinated alcohols useful for the purposes of this invention at 100° C. to 145° C. under atmospheric pressure, the mole ratio of boron to fluorinated alcohol can be in the range between about 0.025 to 1.0. Alternatively, this range can be of about 0.01 to 0.10.

The bromine or iodine compound useful for the disclosed method is of the general Formula (3)

MX (3)

wherein M is a cation of the alkali metals Na⁺, K⁺, Li⁺ or a cation of the type R²R³R⁴R⁵N⁺ or R²R³R⁴R⁵P⁺ where R², R³, R⁴, and R⁵ independently are hydrocarbyl groups of 1 to 20 carbon atoms as described in the commonly owned and co-pending U.S. Patent Publications 2010/0280278 and 2010/0279852 which are herein incorporated by reference in their entirety. Typically, R², R³, R⁴, and R⁵ independently are alkyl groups of 1 to 4 carbons, such as butyl, and can be the same or different. In one embodiment, M is R²R³R⁴R⁵N⁺; X is bromide, or iodide, but is typically iodide; MX may be a mixture of MX compounds, for instance a mixture of NaI and Bu₄NI may be used in combination with a boron compound to provide a catalyst for the preparation of the fluorinated alkoxylates of the present invention.

Inert materials or solvents can be also present during the reaction. In a preferred embodiment the fluorinated alcohol or alcohol mixture is contacted in neat form with the alkoxylating agent in the presence of the catalytic system. Additionally, the fluorinated alcohol be thoroughly dried, using methods known to those skilled in the art, prior to reaction with the alkoxylating agent to avoid undesirable side reactions.

In one specific embodiment of the disclosed method the fluorinated alkylalkoxylates of Formula (1) defined above can be prepared by the reaction of a fluorinated alcohol having the general structure of Formula (2), R_{f}O(CF₂)ₓ(CH₂)_{y}OH, as defined above, with ethylene oxide in the presence of the above described catalyst in accordance with the following equation:

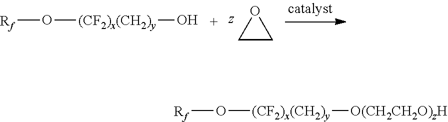

The fluoroalcohols used as starting materials to make the compositions of the present invention are available by the following series of reactions:

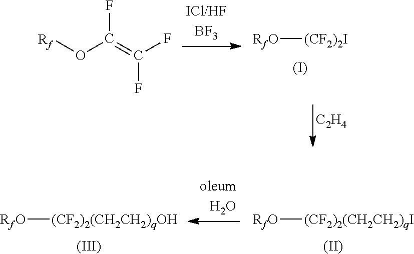

The starting perfluoroalkyl ether iodides of formula (I) above can be made by the procedure described in Example 8 of U.S. Pat. No. 5,481,028, herein incorporated by reference, which discloses the preparation of compounds of formula (I) of perfluoro-n-propyl vinyl ether.

In the second reaction of the reaction (preparation of formula (II) series shown directly above, a perfluoroalkyl ether iodide (I) is reacted with an excess of ethylene at an elevated temperature and pressure. The addition of ethylene can be carried out thermally. Alternatively a suitable catalyst can be used. The catalyst can be a peroxide catalyst such as benzoyl peroxide, isobutyryl peroxide, propionyl peroxide, or acetyl peroxide. Alternatively, the peroxide catalyst can be benzoyl peroxide. The temperature of the reaction is not limited and can be in the range of 110° C. to 130° C. The reaction time can vary with the catalyst and reaction conditions, but 24 hours is usually adequate. The product is purified by any means that separate unreacted starting material from the final product, but distillation is preferred. Satisfactory yields up to 80% of theory have been obtained using about 2.7 mols of ethylene per mole of perfluoalkyl ether iodide, a temperature of 110° C. and autogenous pressure, a reaction time of 24 hours, and purifying the product by distillation.

The perfluoroalkylether ethylene iodides (formula II) are treated with oleum and hydrolyzed to provide the corresponding alcohols (formula III) according to procedures disclosed in WO 95/11877 (Elf Atochem S. A.). Alternatively, the perfluoroalkylether ethyl iodides can be treated with N-methyl formamide followed by ethyl alcohol/acid hydrolysis. The temperature can be of about 130° to 160° C. The higher homologs (q=2, 3) of telomer ethylene iodides (II) are available with excess ethylene at high pressure.

Specific fluoroether alcohols useful in forming compounds of the invention include those listed in Table 1. The groups C₃F₇, C₄F₉, and C₆F₁₃, in the list of specific alcohols in Table 1, refer to linear perfluoroalkyl groups unless specifically indicated otherwise.

TABLE 1
Fluoroether alcohols useful in forming compounds of the invention

F₃COCF₂CF₂CH₂CH₂OH,
F₃CO(CF₂CF₂)₂CH₂CH₂OH,
C₂F₅OCF₂CF₂CH₂CH₂OH,
C₂F₅O(CF₂CF₂)₂CH₂CH₂OH,
C₃F₇OCF₂CF₂CH₂CH₂OH,
C₃F₇O(CF₂CF₂)₂CH₂CH₂OH,
C₄F₉OCF₂CF₂CH₂CH₂OH,
C₄F₉O(CF₂CF₂)₂CH₂CH₂OH,
C₆F₁₃OCF₂CF₂CH₂CH₂OH,
C₆F₁₃O(CF₂CF₂)₂CH₂CH₂OH,
F₃COCF(CF₃)CF₂OCF₂CF₂CH₂CH₂OH,
F₃COCF(CF₃)CF₂O(CF₂CF₂)₂CH₂CH₂OH,
C₂F₅OCF(CF₃)CF₂OCF₂CF₂CH₂CH₂OH,
C₂F₅OCF(CF₃)CF₂O(CF₂CF₂)₂CH₂CH₂OH,
C₃F₇OCF(CF₃)CF₂OCF₂CF₂CH₂CH₂OH,
C₃F₇OCF(CF₃)CF₂O(CF₂CF₂)₂CH₂CH₂OH

The following equipment and test method were used in the Examples herein.

### EXAMPLES

### General Methods, Equipment and Materials Equipment

A 250 milliliters (mL) round bottom flask (RBF) was used as a reactor for alkoxylation reactions at atmospheric pressure. The flask was equipped with a gas inlet tube connected to an ethylene oxide (EO) feed line, a dry ice condenser, and a mechanical agitator. A thermocouple connected to a J-KEM, Gemini controller (from J-KEM Scientific, Inc., St. Louis, Mo.) was used to control batch temperature.

The ethylene oxide (EO) feed line included a 2.27 kg ethylene oxide cylinder, mounted on a lab balance. The ethylene oxide cylinder was equipped with an exterior shut-off gate valve and connected in series with a check valve and a needle control valve. This EO feed was via a T-line connected to a dry nitrogen flow to allow a mixture of nitrogen and EO to enter the reactor. A dry trap was inserted just before the reactor to buffer the EO feed line against unanticipated reactor back flow. Flow was monitored by a gas bubbler filled with KRYTOX, available from E. I. du Pont de Nemours and Company, Wilmington, Del., and two rotometers in line with both the nitrogen and the EO individually.

A scrubber system included an exit line from the dry ice condenser. The exit line passed through a KRYTOX exit bubbler and then through two scrubber bottles; one dry bottle to act as a buffer between the reactor and the scrubber and, the second scrubber was filled with 10% aqueous sodium hydroxide.

Ethoxylation reactions at elevated pressure were performed in a stainless steel reactor. In some cases a glass liner was used. The reactor was charged with the alcohol, a magnetic stir bar, catalyst components, and then connected to a gas manifold. The reactor was evacuated and then a premeasured amount of EO, in a ratio of EO/alcohol typically of about 4 to 12, was condensed into the reactor at 0-5° C. When the EO transfer was complete the system was backfilled with ca. 1 psig nitrogen and the feed valves closed. The reactor was placed in a block heater and brought to reaction temperature and stirred magnetically. Reaction progress was followed by monitoring the pressure. At the higher catalyst concentrations (ca. 6 mole %) gas uptake was normally complete within 3-6 hours. Lower catalyst concentrations required longer times and were typically allowed to proceed overnight to ensure complete ethylene oxide consumption.

For analysis and work up the reactor was cooled to 0-3° C. with ice. Unreacted EO, if present, was removed by vacuum and collected in a −196° C. trap. The ethoxylate product was analyzed by Gas Chromatography (GC) and various other techniques (HPLC, MS, NMR).

### Test Method 1—Surface Tension Measurement

Surface tension was measured using a Kruess Tensiometer, K11 Version 2.501 in accordance with instructions with the equipment. The Wilhelmy Plate method was used. A vertical plate of known perimeter was attached to a balance, and the force due to wetting was measured. Samples to be tested were diluted with water. Each Example was added to deionized water by weight based on solids of the additive in deionized water; Standard Deviation was less than 1 dynes/cm (1 mN/m); Temperature was about 21° C. Normal surface tension of deionized water is 72-73 dynes/cm (72-73 mN/m). Ten replicates were tested of each dilution, and the following machine settings were used: Method: Plate Method SFT; Interval: 1.0 second (s); Wetted length: 40.2 millimeter (mm); Reading limit: 10; Min Standard Deviation: 2 dynes/cm (2 mN/m); Gr. Acc.: 9.80665 m/s².

### Material and Test Methods

The following material and test methods were used in the examples herein.

C₃F₇OCF₂CF₂I was made using the method described in U.S. Pat. No. 5,481,028 which is herein incorporated by reference. Benzoyl peroxide, N-methyl-formamide and Tetrabutylammonium iodide were obtained from Sigma-Aldrich, Milwaukee, Wis. Perfluoropropylvinlyether was from DuPont Co., Wilmington, Del. Ethylene oxide was from GT&S, Inc., Allen Town, Pa.

### Floor Polish

RHOPLEX® 3829, formulation N-29-1, from Rohm & Haas (Spring House, Pa.) was used.

### Test Methods

### Test Method 1—Surface Tension Measurements

The surface tension of the samples in a floor polish (RHOPLEX® 3829, Formulation N-29-1) was measured via a Kruess Tensiometer, K11 Version 2.501. The Wilhelmy Plate method (which is well known in the art) was used. A vertical plate of known perimeter was attached to a balance, and the force due to wetting was measured. Ten replicates were tested of each dilution, and the following machine settings were used:
Method: Plate Method SFT
Interval: 1.0 s
Wetted length: 40.2 mm
Reading limit: 10
Minimum Standard Deviation: 0.1 dynes/cm
Gr. Acc.: 9.80665 m/ŝ2

### Test Method 2—Wetting/Leveling Test

To test the performance of the samples in their wetting and leveling ability, the samples were added to a floor polish (RHOPLEX® 3829, Formulation N-29-1) and applied to half of a stripped 12 inch (30.48 centimeters) by 12 inch vinyl tile. Following the resin manufacturer protocols, a 1 percent (%) (active ingredient basis) solution was prepared by dilution in deionized water. The formulation, as prepared, required a 0.75% (weight basis) of the 1% surfactant dilution.

The floor polish was applied to the tile by pippetting 3 mL of the polish in the center of the tile, and then was spread from top to bottom using a folded piece of cheesecloth. A large “X” was place across the tile, using the cheesecloth. The tile was allowed to dry for 30 minutes (min) and a total of 5 coats were applied. After each coat, the tile was rated on a 1 to 5 scale (1 being the worst, 5 the best) on the surfactant's ability to promote wetting and leveling of the polish on the tile surface. The rating was determined based on comparison of a tile treated with the floor polish that contained no fluorosurfactant.

### Syntheses

### Synthesis Polyfluoroether Alcohol [C₃F₇OCF₂CF₂CH₂CH₂OH]

C₃F₇OCF₂CF₂I (100 grams (g), 0.24 mole (mol)) and benzoyl peroxide (3 g) were charged to a pressure vessel under nitrogen. A series of three vacuum/nitrogen gas sequences was then executed at −50° C. and ethylene (18 g, 0.64 mol) was introduced. The vessel was heated for 24 hours (h) at 110° C. The autoclave was cooled to 0 degrees Celsius (° C.) and opened after degassing. Then the product was collected in a bottle. The product was distilled giving 80 g of C₃F₇OCF₂CF₂CH₂CH₂I in 80% yield. The boiling point was 56˜60° C. at 25 millimeters Mercury (mm Hg, 3333 Pa).

A mixture of C₃F₇OCF₂CF₂CH₂CH₂I (300 g, 0.68 mol) and N-methyl-formamide (300 mL), was heated to 150° C. for 26 h. Then the reaction was cooled to 100° C., followed by the addition of water to separate the crude ester. Ethyl alcohol (77 mL) and p-toluene sulfonic acid (2.59 g) were added to the crude ester, and the reaction was stirred at 70° C. for 15 min. Then ethyl formate and ethyl alcohol were distilled out to give a crude product. The crude product was dissolved in ether, washed with aqueous sodium sulfite, water, and brine in turn, then dried over magnesium sulfate. The product was then distilled to give 199 g of C₃F₇OCF₂CF₂CH₂CH₂OH in 85% yield. The boiling point was 71˜73° C. at 40 mm Hg (5333 Pa).

### Synthesis of B(OCH₂CH₂CF₂CF₂OCF₂CF₂CF₃)₃

A flask was charged with B(OH)₃, 3.02 molar equivalents of HOCH₂CH₂CF₂CF₂OCF₂CF₂CF₃ and toluene. The mixture was brought to reflux and the water evolved was removed with a Dean-Stark trap as is well-known in the art. When water removal was complete the toluene was removed under vacuum to yield the product as a colorless liquid in 70% yield. ¹H NMR (CDCl3): 4.09 (t, 2H), 2.28 (m, 2H).

### Examples 1

### Preparation of Ethoxylate of Perfluoroalkylethers with Varying Linkages

Four separate samples with varying linkages were prepared as described herein. For each reaction, a 250 mL capacity four-necked round-bottomed flask, equipped with a mechanical stirrer, a dry ice condenser and a gas inlet was charged with 40 g of perfluoropropylvinly-ether alcohol (PPVE alcohol), 0.4 g of sodium iodide, and 0.2 g of sodium borohydride, under an inert atmosphere and 1 atmosphere pressure. Each reactor was continuously purged with nitrogen and dry ice was added to the condenser. Each reactor was wrapped with fiberglass cloth insulation to reduce heat loss and minimize light intrusion. The content of each reactor was then heated up to 125±5° C. after the addition of the catalyst and the initiator and held at that temperature for 1 h under constant stirring. After 1 h, ethylene oxide (EO) was fed batchwise at the rate of approximately 6 grams per hour (g/h). The rate of EO addition was adjusted based on the weight loss recorded by a digital balance on which the EO cylinder was placed as a function of time. EO was turned off when the reflux became heavy. EO valve was turned on, once again, when there was little or no reflux. A total of ca 31.4 g of EO was added maintaining the reaction temperature at 125±5° C. After ca 31.4 g EO was added, dry ice was removed from the condenser and the reaction mixture was purged with nitrogen for an h at 125±5° C. to remove any residual EO. The reactor was then cooled down and 20 g of the reactor content was transferred into sample bottle for work up and analysis. The sample was analyzed by NMR to determine the number of EO units added to the PPVE alcohol and has been listed in the Table 2 below.

Subsequently, the same procedure was repeated with the remaining reactor content and additional 4.2, 4.4, and 4.4 g of ethylene oxide was added, respectively, after removing 20 g of sample before adding EO each time. The results of EO addition are shown in Table 2 below.

TABLE 2
Number of EO Units Added to PPVE Alcohol

| Sample | Average number of EO units added |
|---|---|
| 1 | 5.9 |
| 2 | 6.7 |
| 3 | 8.4 |
| 4 | 10 |

### Example 2

### Preparation of Ethoxylate of Perfluoroalkylether

A pressure reactor was charged with HOCH₂CH₂CF₂CF₂OCF₂CF₂CF₃, 6 mole % B(OCH₂CH₂CF₂CF₂OCF₂CF₂CF₃)₃, and 6 mole % tetrabutylammonium iodide. Excess EO was added to the reactor, which was then heated to 110° C. Pressure drop, indicative of ethylene oxide uptake, was evident at 100-105° C. The reaction was stopped when ethylene oxide uptake was complete (final P=101 KPa) and the opaque liquid product was collected. GC analysis showed 99.5% alcohol conversion to a narrow distribution of ethoxylated product averaging 8 moles of EO per alcohol, e.g., C₃F₇OCF₂CF₂CH₂CH₂O(CH₂CH₂O)₈H.

### Performance Surface Tension and Leveling

Samples of the PPVE ethoxylate from Example 1 were evaluated for surface tension reduction in water and Rhoplex as described in the procedures above and the results are summarized in Tables 3 and

TABLE 3
Surface Tension in DI Water (dynes/cm)

| Sample* | 0.000% | 0.001% | 0.010% | 0.100% | 0.500% |
|---|---|---|---|---|---|
| 1 | 73.3 | 40.4 | 25.9 | 18.7 | 17.1 |
| 2 | 72.6 | 51.9 | 33.7 | 17.9 | 17.7 |
| 3 | 73.6 | 46.3 | 32.7 | 18.0 | 18.1 |
| 4 | 74 | 48.3 | 34.2 | 18.1 | 18.0 |

*Samples were added to deionized water by weight based on solids of the additive in deionized water (DI water).
*Standard Deviation <1 dynes/cm
*Temperature 23° C.

Normal surface tension of deionized water is 72 dyne/cm (shown in Table 2 as 0.000%). When each sample was added at a specified rate, the surface tension of each aqueous solution was reduced significantly. Better performance was obtained at higher levels. According to the results from these tests, excellent surface tension reduction was seen from all sample tested.

TABLE 4
Surface Tension in Rhoplex (dynes/cm)

| Sample* | 0.000% | 0.001% | 0.010% | 0.100% | 0.500% |
|---|---|---|---|---|---|
| 1 | 33.9 | 32.9 | 30.1 | 28.5 | 23.2 |
| 2 | 33.4 | 31.9 | 32.0 | 29.0 | 25.0 |
| 3 | 34.1 | 33.0 | 30.0 | 26.0 | 24.1 |
| 4 | 33.4 | 31.9 | 32.0 | 29.0 | 25.0 |

*Samples were added to deionized water by weight based on solids of the additive in the floor polish.
*Standard Deviation <1 dynes/cm
*Temperature 23° C.

Normal surface tension of RHOPLEX® is 34 dyne/cm (shown in Table 3 as 0.000%). When each sample was added at a specified concentration, the surface tension of each aqueous solution was reduced significantly. Better performance was obtained at higher levels. According to the results from these tests, excellent surface tension reduction was seen from all samples tested.

### Comparative Sample

In this comparative study, the ethoxylate of a perfluoroalkylethyl alcohol mixture of the formula F(CF₂)ₐCH₂CH₂OH, wherein (a) ranged from 6 to 14, and was predominately 6, 8, and 10 was prepared. The typical mixture was as follows: 27% to 37% of a=6, 28% to 32% of a=8, 14% to 20% of a=10, 8% to 13% of a=12, and 3% to 6% of a=14. The procedure described in Example 1 was employed to prepare the ethoxylate of this alcohol mixture. The average EO number was 9.4. Surface tension reduction with this product was evaluated in deionized water and floor polish. Surface tension results are shown in Tables 5 and 6. The product was added to deionized water and floor polish in an amount of 0.75% (weight basis) of 1% the surfactant dilution and tested for leveling using Test Method 2 described above. Leveling results are shown in Table 7.

TABLE 5
Surface Tension in DI Water (dynes/cm)

| | 0.000% | 0.001% | 0.010% | 0.100% | 0.500% |
|---|---|---|---|---|---|
| Comparative | 73.2 | 54.1 | 25.7 | 23.7 | 22.6 |
| Sample | | | | | |

*Sample was added to deionized water by weight based on solids of the additive in DI water.
*Standard Deviation <1 dynes/cm

TABLE 6
Surface Tension in Rhoplex (dynes/cm)

| | 0.000% | 0.001% | 0.010% | 0.100% | 0.500% |
|---|---|---|---|---|---|
| Comparative | 33.6 | 31.4 | 30.8 | 28.7 | 25.2 |
| Sample | | | | | |

*Sample was added to deionized water by weight based on solids of the additive in the floor polish.
*Standard Deviation <1 dynes/cm
*Temperature 23° C.

TABLE 7
Leveling in Rhoplex Floor Finish

| | | Ethoxylate | Ethoxylate from |
|---|---|---|---|
| Coating | | from Example | Comparative |
| # | Blank | 1, Sample 4 | Sample |
| 1 | 2 | 2 | 2 |
| 2 | 1 | 3 | 2.5 |
| 3 | 1 | 3.5 | 3 |
| 4 | 2.5 | 4 | 4 |
| 5 | 3 | 3.5 | 4 |
| Average | 1.90 | 3.20 | 3.10 |

These surfactants also exhibited excellent wetting ability in a floor finish (RHOPLEX®) formulation. The compounds performed equal to or better than comparative sample comprising fluorinated ethoxylate having longer perfluorinated alkyl groups when tested on vinyl tile. These surfactants show significant improvement over the “blank” sample where no fluorinated surfactants were used.

What is claimed is:

1. A compound comprising Formula 1,
R_{f}—O—(CF₂)ₓ(CH₂)_{y}—O—(QO)_{z}—H (1)
wherein
R_{f} is a linear or branched perfluoroalkyl having 1 to 6 carbon atoms optionally interrupted by one to three ether oxygen atoms;
x is an integer of 1 to 6;
y is an integer of 1 to 6;
Q is a linear 1, 2-alkylene group of the formula CₘH₂ₘ where m is an integer of 2 to 10; and
z is an integer of 1 to 30.

2. The compound of claim 1 wherein R_{f} is C₃F₇.

3. The compound of claim 1 wherein R_{f} is CF₃CF₂CF₂.

4. The compound of claim 1 wherein x is 2.

5. The compound of claim 1 wherein y is 2.

6. The compound of claim 1 wherein Q is selected from the group consisting of CH₂CH₂, CH₂CH(CH₃), and mixtures thereof.

7. A composition comprising a mixture of compounds of Formula 1 of claim 1 wherein the mixture of compounds of Formula 1 has varying amounts of z in the range of 4 to 15.
